(19) United States
(12) Patent Application Publication
(10) Pub. No.: US 2025/0170048 A1
(43) Pub. Date: May 29, 2025

(54) EMOLLIENT COMPOSITION

(71) Applicant: CARGILL, INCORPORATED, Wayzata, MN (US)

(72) Inventors: Jeffrey Pearson Addy, Goodyear, AZ (US); Frédéric Niola, Vitry sur Seine (FR)

(73) Assignee: CARGILL, INCORPORATED, Wayzata, MN (US)

(21) Appl. No.: 18/839,797

(22) PCT Filed: Feb. 28, 2023
(86) PCT No.: PCT/US2023/063395

§ 371 Date: Aug. 20, 2024

Related U.S. Application Data
(60) Provisional application No. 63/268,640, filed on Feb. 28, 2022.

(51) Int. Cl. A61K 8/92; A61K 8/37; A61Q 5/12; A61Q 17/04; A61Q 19/00

(52) U.S. Cl. CPC A61K 8/922; A61K 8/37; A61Q 5/12; A61Q 17/04; A61Q 19/00

(57) ABSTRACT

The disclosure relates to emollient compositions and a method of making the same. The emollient composition comprises a mixture of cocoa butter fatty acid ethyl esters. The method comprises the steps of: (a) providing cocoa butter and ethyl alcohol; and (b) mixing the cocoa butter and the ethyl alcohol in the presence of an esterification catalyst at an esterification temperature to obtain the cocoa butter fatty acid ethyl esters. A personal care product comprising the emollient composition is also disclosed herein.

### CROSS-REFERENCE TO RELATED APPLICATIONS

This application claims the benefit of U.S. Provisional Patent Application No. 63/268,640, filed Feb. 28, 2022, which is hereby incorporated by reference in its entirety.

### FIELD OF INVENTION

The present invention relates to emollient compositions, methods of making the same, and their use in personal care products.

### BACKGROUND

An emollient is a personal care product commonly used for relieving skin dryness by keeping skin moist. Emollients can be a wide spectrum of various ingredients, from synthetically derived compounds (e.g., mineral oils), to natural components (e.g., vegetable oils).

Mineral oils, byproducts of petroleum refining, have long been around as a major component in emollient formulations due to its low volatility and smoothing texture. Like other moisturizers, mineral oils help to lock in moisture to sooth dry and irritated skin. However, since mineral oils do not readily absorb into and penetrate the skin, they remain on the skin surface and can clog pores, causing other problems to the skin.

There have been numerous efforts in the personal care and cosmetic industries to develop a bio-based and natural alternative to mineral oils and vegetable oils have appeared as one of the most popular alternatives. A key benefit of vegetable oils is their natural source. They are generally not harmful to the environment and more closely mimic the lipid composition of the skin and hair, making them easy absorbed. They promote protection of the skin against excessive loss of water and nutrients, without interfering with the natural functions of the skin, and help in moisturizing and structuring of the underlying tissues.

Despite these desirable characteristics, natural vegetable oils have some inherent limitations. Oxidation of the vegetable oils, together with degradation of the antioxidants, could end up causing the product to acquire undesirable characteristics, and even to lose its effect after a relatively short shelf life.

Cocoa butter is the fat derived from cocoa beans. It is widely accepted and recognized as an essential ingredient for making chocolate and confectioneries. With a pleasing fatty acid profile and a good smell of the skin, cocoa butter has emerged as one of the most promising and healthy ingredients in cosmetic and person care products.

Since there is a growing demand for naturally based ingredients useful in personal care and cosmetic applications, it would be advantageous to have novel and natural based materials with improved properties that more closely mimic the texture, appearance, stability, and properties of conventional materials for making person care and cosmetic products. It would also be desirable for such novel materials to be more oxidatively stable.

### SUMMARY

The present disclosure provides an emollient composition comprising a mixture of cocoa butter fatty acid ethyl esters. Also described herein is a personal care formulation comprising the emollient composition. The present disclosure further provides a method of making a composition comprising cocoa butter fatty acid ethyl esters. The method comprises the steps of: (a) providing cocoa butter and ethyl alcohol; (b) mixing the cocoa butter and the ethyl alcohol in the presence of an esterification catalyst at an esterification temperature to obtain the cocoa butter fatty acid ethyl esters.

### DETAILED DESCRIPTION

Explanations of abbreviations and terms used in this disclosure are provided to assist in comprehending and practicing the invention.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

All percentages of composition or formulation components referred herein are disclosed as percentages by weight (wt %), unless otherwise specified.

All parameter ranges disclosed include the end-points and all values in between, unless otherwise specified.

When used in this specification and claims, the terms “comprise” and “comprising” and variations thereof mean that the specified features, steps, or integers are included. The terms are not to be interpreted to exclude the presence of other features, steps, or components.

As used herein, the singular forms “a,” “an,” and “the” and similar referents in the context of describing the elements (especially in the context of the following claims) include plural referents unless the context clearly dictates otherwise. For example, reference to “a substituent” encompasses a single substituent as well as two or more substituents, and the like. It is understood that any term in the singular may include its plural counterpart and vice versa, unless otherwise indicated herein or clearly contradicted by context.

The term “or” as used herein refers to a nonexclusive “or” unless otherwise indicated. The statement “at least one of A and B” has the same meaning as “A, B, or A and B.”

The terms “for example,” “for instance,” “such as,” or “including” as used herein are meant to introduce examples that further clarify more general subject matter. Unless otherwise specified, these examples are provided only as an aid for understanding the applications illustrated in the present disclosure and are not meant to be limiting in any fashion.

The term “about” as used herein can allow for a degree of variability in a value or range, for example, plus or minus within 10%, within 5%, or within 1% of a stated value or of a stated limit of a range and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%.

In the methods described herein, the acts can be carried out in any order without departing from the principles of the disclosure, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

Representative features are set out in the following description, which stand alone or may be combined, in any combination, with one or more features disclosed elsewhere in the description and/or drawings of the specification.

The term “fatty acid” as used herein refers to a molecule comprising a hydrocarbon chain and a terminal carboxylic acid group. As used herein, the carboxylic acid group of the fatty acid may be modified or esterified, for example as occurs when the fatty acid is incorporated into a glyceride or another molecule (e.g., COOR, where R refers to, for example, a carbon atom). Alternatively, the carboxylic acid group may be in the free fatty acid or salt form (i.e., COO″ or COOH). The ‘tail’ or hydrocarbon chain of a fatty acid may also be referred to as a fatty acid chain, fatty acid sidechain, or fatty chain. The hydrocarbon chain of a fatty acid will typically be a saturated or unsaturated aliphatic group. A fatty acid having N number of carbons, will typically have a fatty acid side chain having N-1 carbons.

The term “acylglyceride” as used herein refers to a molecule having at least one glycerol moiety with at least one fatty acid residue that is linked via an ester bond. For example, acylglycerides can include monoacylglycerides, diacylglycerides, triacylglycerides. The group acylglycerides can be further refined by additional descriptive terms and can be modified to expressly exclude or include certain subsets of acylglycerides. For example, the phrase mono- and di-acylglycerides refers to MAGs (monoacylglycerides) and DAGs (diacylglycerides), while the phrase non-MAG/non-DAG acylglycerides refers to a group of acylglycerides which exclude MAGs and DAGs.

A “fatty acid residue” is a fatty acid in its acyl or esterified form.

The levels of particular types of fatty acids may be provided herein in percentages out of the total fatty acid content of an oil. Unless specifically noted otherwise, such percentages are weight percentages based on the total fatty acids, including free fatty acids and esterified fatty acids as calculated experimentally.

A “saturated” fatty acid is a fatty acid that does not contain any carbon-carbon double bonds in the hydrocarbon chain. For example, stearic acid (C18:0) is a saturated fatty acid having an 18-carbon chain without any carbon-carbon double bonds. An “unsaturated” fatty acid contains one or more carbon-carbon double bonds. For example, oleic acid (C18:1) is an unsaturated fatty acid having an 18-carbon chain with one carbon-carbon double bond. A “polyunsaturated” fatty acid contains more than one such carbon-carbon double bond while a “monounsaturated” fatty acid contains only one carbon-carbon double bond. Carbon-carbon double bonds may be in one of two stereoconfigurations denoted cis and trans. Naturally-occurring unsaturated fatty acids are generally in the “cis” form.

Non-limiting examples of fatty acids include C14, C16 (e.g., C16:0, C16:1), C18 (e.g., C18:0, C18:1, C18:2, C18:3, C18:4), C20 (e.g., C20:0, C20:1, C20:2, C20:3, C20:4, C20:5), C22 (e.g., C22:0, C22:1, C22:2, C22:3, C22:4, C22:5, C22:6), and C24 (e.g., C24:0, C24:1, C24:2, C24:3, C24:4, C24:5, C24:6, C24:7) fatty acids. For example, the fatty acids can be myristic (14:0), palmitic (16:0), stearic or isostearic (18:0), oleic (18:1), linoleic (18:2) and α-linolenic (18:3) acids.

The term “C14-C24 fatty acid” as used herein means a fatty acid containing 14-24 carbons. The C14-C24 fatty acid may be straight or branched. In some aspects, the C14-C24 fatty acid is a C16 and/or C18 fatty acid.

The C14-C24 fatty acid may be a mixture of C14-C24 fatty acids.

The term “ethyl ester” means a fatty acid containing 14-24 carbons that is esterified with an ethyl group.

The fatty acid composition of an oil can be determined by methods well known in the art. The American Oil Chemist's Society (AOCS) maintains analytical methods for a wide variety of tests performed on vegetable oils. Hydrolysis of the oil's components to produce free fatty acids, conversion of the free fatty acids to methyl esters, and analysis by gas-liquid chromatography (GLC) is the universally accepted standard method to determine the fatty acid composition of an oil sample. The AOCS Procedure Ce 1-62 describes the procedure used.

The terms “esterification or esterified” means the creation of an ester bond including: 1) the dehydration reaction of an alcohol with an acid; 2) transesterification, the reaction of an alcohol with an ester to form a new ester; or 3) interesterification, the rearrangement of fatty acids within a triacylglycerol structure

A “cocoa butter” as used herein refers to a triacylglyceride (triglyceride or triacylglycerol) derived from a mixture of fatty acids derived from the seeds of the cocoa plant. Non-limiting examples of fatty acids of cocoa butter include C14 (e.g., C14:0), C16 (e.g., C16:0, C16:1), C18 (e.g., C18:0, C18:1, C18:2, C18:3), C20 (e.g., C20:0, C20:1), C22 (e.g., C22:0), and C24 (e.g., C24:0) fatty acids. In one aspect, the cocoa butter has a fatty acid profile that includes, but not limited to, myristic acid (C14:0), palmitic acid (C16:0), palmitoleic acid (C16:1), stearic acid (C18:0), oleic acid (C18:1), linoleic acid (C18:2), α-linolenic (C18:3), arachidic acid (C20:0), paullinic acid (C20:1), behenic acid (C22:0), and lignoceric acid (C24:0).

A “cocoa butter fatty acid ethyl ester” or “cocoa butter ethyl ester (CBEE)” as used herein refers to a cocoa butter fatty acid containing 14-24 carbons (C14-C24 fatty acid) esterified with an ethyl group.

The term “emollient” as used herein refers to a composition or a product that softens, smooths, and moisturizes the skin by building and/or replenishing the skin barrier; by repairing and filling up cracks in the skin; by soothing skin irritation and redness caused by skin dryness and other conditions (e.g., eczema, psoriasis); thus, preventing water loss from skin, relieving skin dryness, protecting the skin, and keeping the skin healthy. An emollient may comprise synthetic compounds (e.g., mineral oil) and/or natural compounds (e.g., vegetable oils). An emollient may be, without limitation, a lotion, a cream, and an ointment depending on the water-to-oil ratio.

In one aspect, the emollient is a vegetable oil and/or a vegetable fat such as cocoa butter. In another aspect, the emollient comprises esters derived from cocoa butter fatty acid that include, but not limited to, cocoa butter fatty acid ethyl esters. In another aspect, the emollient comprises esters derived from cocoa butter fatty acid that include, but not limited to, cocoa butter fatty acid ethyl esters.

The term “personal care formulation or personal care product” means and comprises any cosmetic, hygienic, toiletry, skin care, and topical care products including, without limitation, leave-on products (i.e., products that are left on keratinous substrates after application); rinse-off products (i.e., products that are washed or rinsed from keratinous substrates during or within a few minutes of application); shampoos; hair curling and hair straightening products; hair style maintaining and hair conditioning products; lotions and creams for nails, hands, feet, face, scalp and/or body; hair dye; face and body makeup; nail care products; astringents; deodorants; antiperspirants; anti-acne; antiaging; depilatories; colognes and perfumes; skin protective creams and lotions (such as sunscreens); skin and body cleansers; skin conditioners; skin toners; skin firming compositions; skin tanning and lightening compositions; liquid soaps; bar soaps; bath products; and shaving products.

The texture of such personal care formulation or personal care product is not limited and may be, without limitation, a liquid, gel, spray, emulsion (such as lotions and creams), shampoo, pomade, foam, tablet, stick (such as lip care products), makeup, among others, any of which can be applied to the skin or hair or hair and which typically are designed to remain in contact therewith until removed, such as by rinsing with water or washing with shampoo or soap. Other forms could be gels that can be soft, stiff, or squeezable. Sprays can be non-pressurized aerosols delivered from manually pumped finger-actuated sprayers or can be pressurized aerosols such as mousse, spray, or foam forming formulation, where a chemical or gaseous propellant is used.

The topical formulation comprising the emollient composition disclosed herein may be a cream used for skincare applications.

Formulations prepared using the composition disclosed herein have a white or pale white color that is generally considered to be aesthetically appealing. In some cases, the formulations of the invention may be further processed to make a colored end product. In such cases, the white color is beneficial because it will show up the additional pigment without influencing the final color.

Furthermore, formulations prepared using the composition disclosed herein have an acceptable oil content and good spreadability with less greasy residual feeling on the skin. This texture feels pleasant to touch and apply. Furthermore, the consistency is such that good product pick-up may be achieved. Good product pick-up means that sufficient product (i.e., not too much, and not too little) can be collected on the user's finger.

The composition of the present disclosure is also useful in sunscreen applications. Sunscreens contain ingredients intended to block UV radiation from reaching the skin. UV blockers or sunscreen agents can be physical such as chemical salts like ZnO or TiO2, or chemical (max authorized level indicated) such as avobenzone (3%), homosalate (15%), butyl methoxydibenzoylmethane (5%); octocrylene (10%); titanium dioxide (25%); ethylhexyl salicylate (5%); ethylhexyl methoxycinnamate (10%); bis-ethylhexyloxyphenol methoxyphenyl triazine (10%); composition of the present disclosure can be used with any type of UV blocker or sunscreen agent known in the art or mixtures of UV blockers or sunscreen agents.

### Emollient Composition

The emollient composition described herein comprises a mixture of cocoa butter fatty acid ethyl esters (CBEE). In one aspect, CBEE has a fatty acid ethyl ester profile as shown in Table 1.

TABLE 1
Fatty acid ethyl ester profile of CBEE

| Fatty Acid Ethyl Ester | Weight Percentage (%) |
|---|---|
| Myristic acid (C14:0) ethyl ester | Trace amount |
| Palmitic acid (C16:0) ethyl ester | 25.3 |
| Palmitoleic acid (C16:1) ethyl ester | 0.4 |
| Stearic acid (C18:0) ethyl ester | 36.4 |
| Oleic acid (C18:1) ethyl ester | 32.9 |
| Linoleic (C18:2) ethyl ester | 2.8 |
| A-Linoleic (C18:3) ethyl ester | Trace amount |
| Arachidic acid (C20:0) ethyl ester | 1.2 |
| Paullinic acid (C20:1) ethyl ester | Trace amount |
| Behenic acid (C22:0) ethyl ester | Trace amount |
| Lignoceric acid (C24:0) ethyl ester | Trace amount |

In one aspect, CBEE comprises about 15% to about 30%, about 20% to about 30%, or about 24% to about 27% palmitic acid ethyl ester.

In one aspect, CBEE comprises about 25% to about 40%, about 30% to about 40%, or about 34% to about 37% stearic acid ethyl ester.

In one aspect, CBEE comprises about 25% to about 40%, about 30% to about 38%, or about 31% to about 34% oleic acid ethyl ester.

In one aspect, CBEE comprises about 15% to about 30% palmitic acid ethyl ester, about 25% to about 40% stearic acid ethyl ester, and about 25% to about 40% oleic acid ethyl ester. In one aspect, CBEE comprises about 24% to about 27% palmitic acid ethyl ester, about 34% to about 37% stearic acid ethyl ester, and about 31% to about 34% oleic acid ethyl ester.

In one aspect, CBEE comprises about 55% to about 75%, about 60% to about 70%, about 60% to about 65%, or about 61% to about 63% saturated fatty acid ethyl esters. In one aspect, CBEE comprises about 62% saturated fatty acid ethyl esters.

In one aspect, the emollient composition comprises greater than 50%, greater than 75%, greater than 90%, greater than 95%, or greater than 99% CBEE. In another aspect, content of the CBEE in the emollient composition is a range of about 50% to about 99%, about 75% to about 99%, about 90% to about 99%, about 95% to about 99%, about 95% to about 99.3%, about 95% to about 99.5%, or about 99.3% to about 99.5%. In another aspect, content of the CBEE in the emollient composition is about 99.3% or about 99.5.

In one aspect, the emollient composition can be blended with other emollients, such as natural emollients, synthetic emollients, and/or petroleum-based emollients.

In one aspect, CBEE produced by the method described herein has a transparent liquid or semi-fluidic appearance and a light texture. Further, CBEE is non-sticky with good spreadability, providing a smooth and silky effect. These characteristics make CBEE a good candidate for applications such as personal care and cosmetic products. For example, CBEE can be added in lotions, creams, or skin care products at a loading level of 1-15%. In another example, CBEE can be added in anhydrous balms or sticks at a loading level of 1-30%. In yet another example, CBEE can be added in hair care products at a loading level of 1-10%.

In another aspect, the CBEE has a viscosity, determined by a Brookfield viscometer, of about 19,300 mPa.s.

### Personal Care Formulation

The personal care formulation described herein comprises an emollient composition, in which the emollient composition comprises a mixture of cocoa butter fatty acid ethyl esters (CBEE). In another aspect, CBEE has a fatty acid ethyl ester profile as shown in Table 1.

In one aspect, the personal care formulation described herein can contain any useful amount of the CBEE of the present disclosure. In one aspect, the content of CBEE in the personal care formulation is in a range of 1% to 100%, 10%-50%, 15%-45%, 20%-40%, 22%-38%, or 25%-35% by weight. In one aspect, content of the CBEE in the personal care formulation is 30% by weight.

### Method of Making Cocoa Butter Fatty Acid Ethyl Esters

The method of making cocoa butter fatty acid ethyl esters described herein comprises an esterification step of cocoa butter with ethyl alcohol to obtain an ester mixture.

In one aspect, the method comprises the steps of: (a) providing cocoa butter and ethyl alcohol; and (b) esterifying the cocoa butter with ethyl alcohol in the presence of an esterification catalyst at an esterification temperature to obtain the cocoa butter fatty acid ethyl esters.

Esterification of triglyceride vegetable oils is well known in the art, for example, in the preparation of bio-diesel. Typically, the oil is mixed with an excess of an alcohol(s) and heated to an elevated temperature in the presence of a catalyst. The catalyst needs not be but typically is a metal salt of the corresponding alcohol. The choice of reaction temperature is related to the alcohol utilized and a skilled artisan would choose a temperature to balance the reaction rate with the creation of undesired side products. In one aspect, the esterification temperature is about 70° C., the alcohol is ethanol, and the esterification catalyst is sodium ethoxide.

In one aspect, the method may include additional steps to purify and/or modify the cocoa butter fatty acid ethyl ester. For example, the cocoa butter fatty acid ethyl ester can be desolventized with nitrogen gas and at reduced pressure to remove solvents and volatiles such as ethanol. The desolventized cocoa butter fatty acid ethyl ester can be decanted with nitrogen gas, bleached by a bleaching agent, and deodorized by a deodorizing agent or process. These procedures are also well known in the art of vegetable oil purification and are referred to as “refining”. These steps can be easily modified or varied depending on the impurities sought to be removed.

Unless defined otherwise, all technical and scientific terms used herein have the same meaning as commonly understood by one skilled in the art to which this invention belongs. As used herein, each of the following terms has the meaning associated with it as defined below.

The invention is further described in detail by reference to the following experimental examples. These examples are provided for purposes of illustration only and are not intended to be limiting unless otherwise specified. Thus, the invention should in no way be construed as being limited to the following examples, but rather should be construed to encompass any and all variations which become evident as a result of the teaching provided herein.

### EXAMPLES

### Example 1—Method of Making CBEE

Table 2 shows the ingredients for making CBEE using the method described herein.

TABLE 2
Ingredients for making CBEE

| Components | Composition (%) | Source |
|---|---|---|
| Cocoa butter | 74 | Cargill Incorporated |
| Ethanol (EtOH) | 26 | Anhydrous SDA 40B |
| | | Univar |
| Total | 100 | |

To generate the ethyl ester (i.e., CBEE), cocoa butter was first melted, and an excess of 40% to 50% ethanol and 1.4 wt % (based on the total weight of cocoa butter and ethanol) of 21% sodium ethoxide catalyst in ethanol were mixed with the molten cocoa butter at 70° C. with agitation for 120 minutes, or until the esterification was completed as evident by the ethyl ester produced, which was determined by High Performance Liquid Chromatography (HPLC) analysis.

The CBEE was desolventized under a stream of nitrogen gas and at reduced pressure until the volatiles present in the CBEE was below 0.5% measured by volatiles analyzer (scale-oven). Ethanol removed from the CBEE was recovered. The desolventized CBEE was decanted with nitrogen gas at 70° C. to remove glycerol generated during the esterification. An additional glycerol wash was employed to further motivate any glycerol out of the ethyl ester phase. The decanted CBEE was bleached by absorbent (Sorbsil 92, PQ Corporation; and Perform 6000, Oil-Dri Fluids Purification) at 95° C. under agitation. The bleached CBEE was deodorized by water at 160-165° C. Finally, the deodorized CBEE was cooled to 70° C. and tocopherol (70% tocopherol in vegetable oil base, BASF Covi-Ox T70 EU (non-GM version)) was added to the cooled product.

### Example 2—Oxidative Stability of CBEE

Oxidative stability index (OSI) measures the relative oxidative stability of fatty materials. A sample of CBEE was heated to 110° C. under constant airflow. As oxidative degradation of the material began, volatile short-chain fractions broke away and were entrained in the airflow. The short-chain fractions were then trapped in distilled water and detected by a conductivity probe to provide the OSI reading.

Table 3 below shows the OSI of CBEE and other fatty materials. A value of OSI less than 20 hours is generally considered to be unstable.

TABLE 3
OSI Comparison

| Fatty Acid Ester/ | |
|---|---|
| Silicone | OSI (hours) |
| Dimethicone | 101 |
| CBEE | 79 |
| C12-15 alkyl benzoate | 69 |
| Shea butter ethyl ester | Less than 5 |

As observed, CBEE has an OSI significantly higher than other fatty acid esters and silicones, which are conventional ingredients for making personal care products. The superior oxidative stability enables CBEE to be a good candidate for making personal care products.

### Example 3—Sunscreen Solubility of CBEE

The sunscreen solubility, which measures the solubility of a sunscreen agent in a fatty material, of CBEE were shown in Table 4 below.

TABLE 4
Sunscreen Solubility Comparison

| Fatty Acid Ester/ | Loading Percentage of | Loading Percentage of |
|---|---|---|
| Silicone | Avobenzone (%) | Homosalate (%) |
| CBEE | 15 | 25 |
| C12-15 alkyl benzoate | 20 | 25 |
| Shea butter ethyl ester | Less than 15 | 25 |

Sunscreen solubility was determined through mixing sunscreen agents in a range of loading percentages (10-25%), heating the mixtures up to 70° C., and cooling the heated mixture to room temperature overnight. As observed, CBEE has a sunscreen solubility on conventional sunscreen agents, avobenzone and homosalate, comparable or better than other conventional ingredients for making personal care products. The superior oxidative stability enables CBEE to be a good candidate for making personal care products.

### Example 4—Skin Care Formulation

Table 5 shows a skin care formulation in which CBEE is one of the ingredients.

TABLE 5
Skin Care Formulation

| | Composition | |
|---|---|---|
| INCI Name | (wt %) | Source |
| Sclerotium Gum | 0.40 | Actigum |
| | | CS 11 QD |
| | | Cargill Beauty |
| AQUA (Demineralized Water) | Up to 100 | |
| Glycerine | 2.00 | |
| CBEE | 30.00 | |
| Hydrogenated Sunflower Seed Oil Polyglyceryl-3 | 4.00 | Emulsun |
| Esters (and) Hydrogenated Sunflower Seed Oil | | Floratech |
| Glyceryl Esters (and) Cetearyl Alcohol (and) | | |
| Sodium Stearoyl Lactylate | | |
| Benzyl Alcohol (and) Salicylic Acid (and) | 1.00 | Geogard ECT |
| Glycerin (and) Sorbic Acid | | Arxada |
| Sodium Hydroxide | 0.08 | |

The skin care formulation has a pH adjusted to 5.0-5.5 and an appearance of a viscous white cream. It is stable for 2 months at room temperature and at 45° C.

### Example 5—Skin Care Formulation

Table 6 shows a skin care formulation in which CBEE is one of the ingredients.

TABLE 6
Skin Care Formulation

| | Composition | |
|---|---|---|
| INCI Name | (wt %) | Source |
| Deionized water | 48.00 | |
| Tetrasodium Glutamate Diacetate | 0.10 | Nouryon |
| Glycerin | 5.00 | Cargill |
| Magnesium Aluminum Silicate | 0.80 | Vanderbilt |
| Sclerotium Gum | 0.40 | Cargill |
| Caprylic/Capric Triglyceride | 2.00 | BASF |
| Isononyl Isononanoate | 2.00 | Phoenix |
| | | Chemical |
| CBEE | 10.00 | Cargill |
| Butyl Methoxydibenzoylmethane | 3.00 | EMD |
| Ethylhexyl Salicylate | 5.00 | EMD |
| Homosalate | 10.00 | EMD |
| Ethylhexyl Methoxycrylene | 3.00 | Hallstar |
| Jojoba Oil/Macadamia Seed | 1.00 | Cargill |
| Oil Esters (and) Squalene | | |
| (and) Phytosteryl Macadamiate | | |
| (and) Phytosterols | | |
| Actique Ceramide | 0.10 | Jarchem |
| | | Industries |
| Tocopherol Acetate | 0.25 | Cargill |
| Glyceryl Stearate SE | 6.00 | BASF |
| Potassium Cetyl Phosphate | 0.15 | DSM Nutritional |
| | | Products |
| Polyglycerol-3 Beeswax | 1.80 | Koster Keunen |
| Citric Acid (30%) | 0.40 | Cargill |
| Phenoxyethanol (and) | 1.00 | Schulke & Mayr |
| Ethylhexylglycerin | | |

To prepare the skin care formulation, tetrasodium glutamate diacetate was first added to deionized water in a first vessel with propeller mixing (300-450 rpm) at room temperature. In a second vessel, magnesium aluminum silicate, sclerotium gum, and glycerin were mixed and added to the first vessel with propeller agitation (300-450 rpm) at room temperature. The mixture in the second vessel was then heated to 75-80° C. to obtain a Phase A mixture.

In a third vessel, caprylic/capric triglyceride, isononyl isononanoate, CBEE, ethylhexyl salicylate, homosalate, ethylhexyl methoxycrylene, jojoba oil/macadamia seed oil esters (and) squalene (and) phytosteryl macadamiate (and) phytosterols, actique ceramide, tocopherol acetate, glyceryl stearate SE, potassium cetyl phosphate, and polyglycerol-3 beeswax were mixed with propeller agitation (300-450 rpm). The mixture in the third vessel was then heated to 75-80° C. Butyl methoxydibenzoylmethane was added to the mixture in the third vessel with propeller agitation (300-450 rpm) at 75-80° C. to obtain a Phase B mixture.

The Phase B mixture was added to the Phase A mixture with homomixing agitation (3,000-6,000 rpm) at 75-80° C. until the two mixtures were uniformly blended to obtain a Phase A/B blend. The Phase A/B blend was further mixed with propellor agitation (300-450 rpm) and cooled to 55-60° C.

Citric acid and phenoxyethanol (and) ethylhexylglycerin were added to the cooled Phase A/B blend with propellor agitation (300-700 rpm) at 55-60° C. Mixing was stopped when the temperature of the final mixture reached 45-50° C. to obtain the skin care formulation.

The skin care formulation has a sun protection factor (SPF) of at least 13. SPF was estimated by DSM Sunscreen Optimizer, Broad Spectrum (USA) pass.

### Example 6—Skin Care Formulation

Table 7 shows a skin care formulation in which CBEE is one of the ingredients.

TABLE 7
Skin Care Formulation

| | Composition | |
|---|---|---|
| INCI Name | (wt %) | Source |
| Deionized water | 50.80 | |
| Tetrasodium Glutamate Diacetate | 0.10 | Nouryon |
| Glycerin | 5.00 | Cargill |
| Sclerotium Gum | 0.40 | Cargill |
| Caprylic/Capric Triglyceride | 2.00 | BASF |
| Isononyl Isononanoate | 2.00 | Phoenix |
| | | Chemical |
| CBEE | 10.00 | Cargill |
| Butyl Methoxydibenzoylmethane | 3.00 | EMD |
| Ethylhexyl Salicylate | 5.00 | EMD |
| Homosalate | 10.00 | EMD |
| Jojoba Oil/Macadamia Seed | 1.00 | Cargill |
| Oil Esters (and) Squalene | | |
| (and) Phytosteryl Macadamiate | | |
| (and) Phytosterols | | |
| Actique Ceramide | 0.10 | Jarchem |
| | | Industries |
| Tocopherol Acetate | 0.25 | Cargill |
| Glyceryl Stearate SE | 6.00 | BASF |
| Potassium Cetyl Phosphate | 0.15 | DSM Nutritional |
| | | Products |
| Polyglycerol-3 Beeswax | 1.80 | Koster Keunen |
| Hectorite (and) Hydroxyethylcellulose | 1.00 | Elementis |
| Citric Acid (30%) | 0.40 | Cargill |
| Phenoxyethanol (and) | 1.00 | Schulke & Mayr |
| Ethylhexylglycerin | | |

To prepare the skin care formulation, tetrasodium glutamate diacetate was first added to deionized water in a first vessel with propeller mixing (300-700 rpm). In a second vessel, sclerotium gum and glycerin were mixed with stirring and added to the first vessel with propeller mixing (300-700 rpm). The mixture in the second vessel was then heated to 70-75° C. to obtain a Phase A mixture. The Phase A mixture was homomixed at 70-75° C. to activate sclerotium gum. The resulting mixture was heated to 75-80° C. with homomixing agitation (8,000-8,500 rpm).

In a third vessel, caprylic/capric triglyceride, isononyl isononanoate, CBEE, ethylhexyl salicylate, homosalate, jojoba oil/macadamia seed oil esters (and) squalene (and) phytosteryl macadamiate (and) phytosterols, actique ceramide, tocopherol acetate, glyceryl stearate SE, potassium cetyl phosphate, polyglycerol-3 beeswax, and hectorite (and) hydroxyethylcellulose were mixed and heated to 75-80° C. Mixing continued until the ingredients in the mixture were uniformly blended. When the mixture became liquid, butyl methoxy dibenzoylmethane was added to the mixture to minimize heating time. A Phase B mixture was thus obtained.

The Phase B mixture was added to the Phase A mixture with homomixing agitation (9,000-9,500 rpm) at 70-75° C. to obtain a Phase A/B blend. The Phase A/B blend was mixed by a propeller and cooled to 55-60° C. with propeller agitation (300-450 rpm).

Citric acid was added to the cooled Phase A/B blend at 55-60° C. and phenoxyethanol (and) ethylhexylglycerin was added at 50-55° C. with propeller agitation (500- 700 rpm) to obtain a Phase A/B/C blend. Citric acid thinned out the emulsion, which recovered in the rapid mixing.

Weight of the Phase A/B/C blend was adjusted with warm water at 50-55° C. Mixing was stopped when the temperature of the final mixture reached 45-50° C., or when the final mixture became solid, to obtain the skin care formulation.

The skin care formulation has a sun protection factor (SPF) of at least 13. SPF was estimated by DSM Sunscreen Optimizer, Broad Spectrum (USA) pass.

### Example 7—Skin Care Formulation

Table 8 shows a skin care formulation in which CBEE is one of the ingredients.

TABLE 8
Skin Care Formulation

| | Composition | |
|---|---|---|
| INCI Name | (wt %) | Source |
| Deionized water | 78.44 | |
| Glycerin | 2.00 | Cargill |
| Sclerotium Gum | 0.40 | Cargill |
| CBEE | 15.00 | Cargill |
| Polyglycerol-3 Stearate | 1.75 | Lonza |
| Sodium Stearoyl Lactylate | 0.25 | Kerry |
| Glyceryl Stearate SE | 0.50 | BASF |
| Cetearyl Alcohol | 0.50 | BASF |
| Benzyl Alcohol (and) Salicylic Acid (and) | 1.00 | Lonza |
| Glycerin (and) Sorbic Acid | | |
| Sodium Hydroxide (20% solution) | 0.16 | |

To prepare the skin care formulation, deionized water was heated to 70° C. and glycerin was added to the heated deionized water with agitation (200 rpm) to obtain a Phase A mixture. Sclerotium gum was added to the Phase A mixture with Silverson homogenizer for 5 minutes at 5,000 rpm to obtain a Phase A/B blend.

CBEE, polyglycerol-3 stearate, sodium stearoyl lactylate, glyceryl stearate SE, and cetearyl alcohol were mixed and heated to 70° C. to obtain a Phase C mixture. The Phase C mixture was added to the Phase A/B blend and the resulting mixture was mixed with Silverson homogenizer for 5 minutes at 4,000 rpm to obtain a Phase A/B/C blend. The Phase A/B/C blend was cooled down with propeller agitation (300-600 rpm).

Benzyl alcohol (and) salicylic acid (and) glycerin (and) sorbic acid was added to the cooled Phase A/B/C blend with propeller agitation (300-600 rpm) at 50° C. Sodium hydroxide solution was added to adjust a pH of the obtained skin care formulation to 5.0-5.5.

### Example 8—Skin Care Formulation

Table 9 shows a skin care formulation in which CBEE is one of the ingredients.

TABLE 9
Skin Care Formulation

| | Composition | |
|---|---|---|
| INCI Name | (wt %) | Source |
| Jojoba Esters (saturated esters) | 17.00 | Cargill |
| CBEE | 15.00 | Cargill |
| Jojoba Esters (mixture of diene, | 47.46 | Cargill |
| monoene, and saturated esters) | | |
| Zea mays (corn) starch | 20.00 | Cargill |
| Tocopherol | 0.20 | Cargill |
| Parfum | 0.10 | Aromat'Easy |
| Mica (and) Titanium Dioxide (and) Carmine | 0.24 | BASF |

To prepare the skin care formulation, jojoba esters (saturated esters), jojoba esters (mixture of diene, monoene, and saturated esters), zea mays (corn) starch, tocopherol, parfum, and mica (and) titanium dioxide (and) carmine were heated to 75-80° C. in a water bath. The heated ingredients were mixed with CBEE, which was preheated at 45-50° C. in an oven or a water bath until a transparent homogenous phase was obtained, in a rotor stator mixer (IKA Ultra Turrax T25, S25N-25G workhead) for 1 minute at 6,000 rpm. The mixture was poured into a mould. The moulded mixture was set at room temperature for 5 minutes and stored in a freezer (−18° C.) for 10 minutes, or set at room temperature for 30 minutes before unmolding and packing to obtain the skin care formulation.

### Example 9—Hair Care Formulation

Table 10 shows a hair care formulation in which CBEE is one of the ingredients.

TABLE 10
Hair Care Formulation

| | Composition | |
|---|---|---|
| INCI Name | (wt %) | Source |
| Deionized water | Up to 100 | |
| Glycerin | 1.00 | Cargill |
| Sclerotium Gum | 0.25 | Cargill |
| Hydroxypropyl Starch Phosphate | 4.50 | Cargill |
| Sodium Starch Octenylsuccinate and | 2.00 | Cargill |
| Hydroxypropyl Starch Phosphate | | |
| Cocos Nucifera (Coconut) Oil | 2.00 | Cargill |
| CBEE | 1.00 | Cargill |
| Phenoxyethanol | 1.00 | Schulke & Mayr |
| Tocopheryl Acetate | 0.10 | Zhejiang |
| | | Medicine |
| Synthetic Fluorphlogopite | 0.25 | Sun Chemical |
| & Titanium Dioxide | | |

To prepare the hair care formulation, deionized water, glycerin, sclerotium gum, hydroxypropyl starch phosphate, and sodium starch octenylsuccinate and hydroxypropyl starch phosphate were mixed under Silverson homogenizer at 7,000 rpm for 7 minutes to obtain a Phase A mixture.

Cocos nucifera (coconut) oil and CBEE were mixed in a separate vessle and heated to 50° C. with low mixing until both ingredients were fully melted to obtain a Phase B mixture.

The Phase B mixture was mixed with the Phase A mixture for 10 minutes using a scrapper mixing at 70 rpm to obtain a Phase A/B blend. The Phase A mixture and the Phase B mixture could also be blended manually with a spatula, followed by a higher shear mixing under a Silverson homogenizer at 5,000-7,000 rpm to obtain a Phase A/B blend. The Phase A/B blend was further mixed with a propeller mixer at low mixing (about 800-1,200 rpm) to avoid bubble entrapments.

Phenoxyethanol, tocopheryl acetate, and mica were added to the Phase A/B blend one at a time using a propeller mixer. Deionized water was added to compensate the water lost during the process to obtain the hair care formulation.

The hair care formulation has a pH adjusted to 4.5-5.5 and a viscosity of about 13,500 cP.

In addition to the applications described above, the cocoa butter fatty acid ethyl ester described herein can be used as ingredients of including, but not limited to, skin care products, sun care products, hair care products (e.g., shampoo, conditioner), make-up products, etc.

What is claimed is:

1. An emollient composition comprising a mixture of cocoa butter fatty acid ethyl esters.

2. The emollient composition of claim 1 comprising greater than 90% cocoa butter fatty acid ethyl esters.

3. The emollient composition of claim 1, wherein the cocoa butter fatty acid ethyl esters have about 60% to about 70% saturated fatty acid ethyl esters.

4. A personal care formulation comprising the emollient composition of claim 1.

5. The personal care formulation of claim 4, wherein the content of the cocoa butter fatty acid ethyl ester is between 20% and 40%.

6. The personal care formulation of claim 4 further comprising one or more of the followings: sclerotium gum, glycerin, an emulsifier, a preservative, and sodium hydroxide.

7. The personal care formulation of claim 4 further comprising a sunscreen agent.

8. The personal care formulation of claim 4 further comprising one or more of the followings: a perfume, an active substance, a preservative, and a colorant.

9. The personal care formulation of claim 4, wherein the personal care formulation is selected from a group consisting of a skin protective cream, a lotion, a sunscreen, a skin or body cleanser, a skin conditioner, a skin toner, a shampoo, a hair conditioner, and a skin firming composition.

10. A method of making a composition comprising cocoa butter fatty acid ethyl esters, the method comprising the steps of:
a. providing cocoa butter and ethyl alcohol; and
b. mixing the cocoa butter and the ethyl alcohol in the presence of an esterification catalyst at an esterification temperature to obtain the cocoa butter fatty acid ethyl esters.

11. The method of claim 10, wherein the esterification temperature is about 70° C.
